(19) United States
(12) Patent Application Publication
(10) Pub. No.: US 2003/0165540 A1
(43) Pub. Date: Sep. 4, 2003

(54) Sentinel virus II

(76) Inventors: Jen-Kuei Liu, Palo Alto, CA (US); Samantha Lewis, San Francisco, CA (US); Hans-Georg Batz, Tutzing (DE); Latha Ramaswamy, San Jose, CA (US); Roy Bohenzky, Mountain View, CA (US); Yu-Huei Lin, Sunnyvale, CA (US); Janine Montiel, Fremont, CA (US); Benjamin P. Chen, Fremont, CA (US)

Correspondence Address: Kenneth J. Waite, Roche Diagnostics Corporation, 9115 Hague Road, Bldg. D, P.O. Box 50457, Indianapolis, IN, 46250-0457, US

(21) Appl. No.: 09/849,729

(22) Filed: May 4, 2001

Related U.S. Application Data
(60) Provisional application No. 60/202,271, filed on May 5, 2000.

(51) Int. Cl. A61K 39/29; C12Q 1/70; C07H 21/04; C12N 7/00

(52) U.S. Cl. 424/225.1; 435/5; 536/23.72; 435/235.1

(57) ABSTRACT

The invention relates to a new virus, designated H101.c33 or Sentinel Virus II (SVII). Isolated SVII viruses, polynucleotides and proteins from SVII viruses, and antibodies which bind SVII virus and SVII viral proteins are provided. The polynucleotides, proteins, and antibodies of the invention may be used to detect SVII virus or infection by SVII virus in a susceptible individual. Additionally, polynucleotides of the invention may be inserted into recombinant expression vectors for recombinant production of viral proteins.

### TECHNICAL FIELD

The invention relates to the field of viruses, and particularly to hepatitis viruses.

### BACKGROUND ART

Strictly defined, the term “hepatitis” refers to an inflammation of the liver. A variety of different chemical, viral, and biological agents will induce hepatitis. However, the term hepatitis more commonly refers to an inflammation of the liver caused by a viral infection, particularly a hepatotrophic viral infection.

Viral hepatitis can be divided into two gross categories: acute and chronic. Acute viral hepatitis is characterized by jaundice, malaise, nausea, and elevated blood liver enzymes. Although most cases of viral hepatitis resolve spontaneously, a portion of acute hepatitis victims (generally less than about 10%) develop fulminant necrotizing hepatitis, a disorder with very high morbidity and mortality. Interestingly, many cases of acute hepatitis are so mild as to pass unnoticed or be dismissed as “flu”. Chronic hepatitis gives rise to a much more significant public health problem, and is the most common reason for liver transplant in the United States. Chronic hepatitis is characterized by exacerbations or “flare ups” with symptoms resembling acute hepatitis, as well as portal hypertension and cirrhosis (scarring of the liver) which leads to liver failure. Because acute hepatitis infections can go unnoticed, many chronic hepatitis patients are not diagnosed until their disease is quite advanced, limiting options for treatment.

There are six different families of viruses referred to as “hepatitis viruses” (A, B, C, D, E and G; F having been found to be artifactual). In developed nations, those hepatitis viruses that can establish chronic infections are generally considered to be the most important viruses from a public health standpoint. Of the hepatitis viruses, hepatitis B virus and hepatitis C virus are the only known hepatitis viruses known to establish chronic infections associated with chronic hepatitis. However, HBV and HCV do not account for all cases of transfusion hepatitis. The terms “cryptogenic hepatitis” and “nonA-G” are used to refer to transfusion hepatitis that cannot be attributed to a known hepatitis virus.

Hepatitis B, previously referred to as “transfusion hepatitis” is transmitted via percutaneous, sexual, and vertical routes. The hepatitis B virus, a member of the hepadnaviridae family, can give rise to both acute and chronic hepatitis. The hepatitis B virus (HBV) has been well characterized, and a variety of screening and diagnostic assays are currently available. Additionally, a recombinant vaccine has been created which is currently required for most school age children in the United States.

Hepatitis C, previously known as “non-A, non-B hepatitis” is transmitted primarily via the percutaneous route, although, like HBV, sexual and vertical transmission also occur. Only a minority of acute hepatitis C virus (HCV) infections are clinically apparent, which is problematic because this virus establishes chronic infections at a very high rate. This combination makes chronic HCV infection the leading reason for liver transplant in the United States.

The advent of screening assays for detection of anti-HBV and/or HCV antibodies 2 in donated blood has substantially reduced the transmission of “transfusion hepatitis.” However, 20-30% of infectious blood donations still go undetected. The failure to detect 4 these infectious samples is believed to be largely due to the existence of one or more, as yet unidentified, hepatitis virus.

More recently, in addition to the six known hepatitis viruses, new, hepatitis-associated viruses have been identified. The virus known as TTV was first identified by 8 a Japanese group, who identified genomic sequences from the virus using representation difference analysis (RDA) technology (Nishizawa et al., 1997, Biochem. Biophys. Res. Commun, 241(1):92-97). This virus, which was originally believed to be a member of the parvoviridae family, is a relatively small virus with a buoyant density significantly lower than that of parvoviridae. TTV has been proposed as the prototypic human member of a family of viruses known as the circinoviridae for their circular, single-stranded DNA genomes (Mushahwar et al., 1999, Proc. Natl. Acad. Sci. USA, 96(6):3177-3182).

Recently, Diasorin, Inc. has announced the isolation of a new hepatitis virus. The virus, termed SEN-V, was later found to be highly prevalent in the healthy population and not limited to blood samples from hepatitis patients and is unlikely a hepatitis virus. Neither the polynucleotide sequence nor methods for isolation of SEN-V have been disclosed.

Accordingly, there is a need in the art for compositions and methods for detection of non-A/non-G hepatitis, as well as compositions and methods for prevention of non-A/non-G hepatitis infections and compositions and methods for treatment of non-A/non-G hepatitis infections.

### DISCLOSURE OF THE INVENTION

We have discovered a new virus associated with cryptogenic, non-A/non-G hepatitis. A variety of valuable inventions have been derived therefrom, providing, for example:

1) Compositions comprising isolated SVII virus. Examples of isolated SVII viruses include isolated viruses comprising the polynucleotide sequence of FIG. 1.

2) Isolated polynucleotides including an isolated polynucleotide selectively hybridizable with the nucleotide sequence of FIG. 1 and complements thereof, an isolated polynucleotide encoding an isolated SVII protein or fragment thereof and complements thereof. The isolated polynucleotide may be an antisense polynucleotide.

3) Compositions comprising an isolated SVII protein or fragment thereof.

4) Vaccine compositions comprising an isolated SVII protein or fragment thereof. The vaccine compositions may include a pharmaceutically acceptable excipient and/or an adjuvant.

5) Expression vectors comprising an isolated polynucleotide encoding a SVII protein or fragment thereof.

6) Expression vectors comprising an isolated polynucleotide, wherein transcription of said isolated polynucleotide results in the production of an SVII antisense polynucleotide.

7) Isolated polyclonal antibodies and monoclonal antibodies which bind to a SVII virus or a protein thereof.

8) Methods for detecting SVII virus, comprising contacting a sample with an antibody which binds to SVII virus or a protein thereof; and detecting complexes of said antibody and SVII virus or protein thereof.

9) Methods for detecting SVII virus, comprising contacting a sample with a probe polynucleotide which selectively hybridizes to a SVII polynucleotide and detecting hybridization of said probe with a SVII polynucleotide.

10) Methods for detecting SVII virus, comprising contacting a sample with a first primer polynucleotide that selectively hybridizes with a SVII polynucleotide and a second primer polynucleotide that hybridizes with a complement of the SVII polynucleotide, performing primer extension DNA synthesis, and detecting the product of the synthesis.

### BRIEF DESCRIPTION OF THE DRAWINGS

FIG. 1 shows nucleotide sequence and conceptual translations of open reading frames from a Sentinel Virus II (SVII) clone (standard single letter coding is employed; k, m, r, s, y and w indicate T/G, A/C, G/A, G/C, T/C and A/T, respectively). The strand designated “positive” is labeled “+”, and its inverse complement is labeled “−”. Numbering refers to the + strand nucleotide sequence. Open reading frames P1, M1 and M2 are also shown. The − strand and the M1 and M2 reading frames are read right to left.

### DETAILED DESCRIPTION OF THE INVENTION

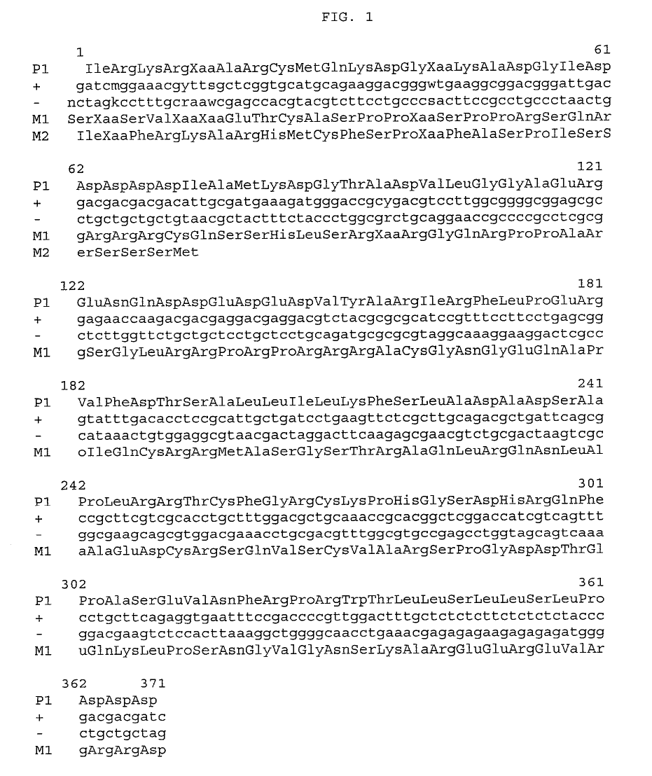

We have discovered and isolated a new hepatitis virus, designated Sentinel Virus II (SVII), that is associated with cryptogenic, nonA-G hepatitis. The prototypic virus comprises a DNA genome of at least about 371 bases. Genomic sequence from the prototypic virus is shown in FIG. 1. Accordingly, the invention provides isolated SVII.

In one aspect, the invention provides isolated polynucleotides comprising the SVII viral genome and fragments thereof. The polynucleotides may be DNA or RNA. Also provided are isolated nucleotide probes or primers for use in detecting SVII infections and/or SVII virus itself. The probes and/or primers may also be used in methods for identification and isolation of new variants of SVII.

A further aspect of the invention provides isolated SVII viral proteins and/or fragments thereof, as well as fusion proteins comprising a SVII viral protein or fragment thereof fused with a heterologous (non-SVII) protein. Also included are mosaic polypeptides that comprise at least two SVII epitopes. In mosaic polypeptides of the invention comprising two epitopes from the same SVII protein, the intervening amino acids between the epitopes are substantially deleted or substituted with a heterologous sequence. Alternately, mosaic polypeptides of the invention may comprise two epitopes from different SVII proteins or comprise homologous epitopes from at least two viruses of the SVII family.

The invention provides recombinant expression constructs, comprising a polynucleotide sequence derived from an open reading frame of an SVII virus operably linked to promoter operable in a prokaryotic or eukaryotic host cell. Also provided are expression vectors and recombinant host cells comprising the expression constructs.

Also provided are antibodies specific for epitopes of the SVII family of viruses. Included are monoclonal antibodies and isolated polyclonal antibodies.

In another aspect, the invention provides assays and kits for conducting assays for detection of SVII infection and/or detection of SVII virus. The assays of the invention may be immunoassays utilizing polypeptides or antibodies of the invention or nucleic acid-based assays employing hybridization or amplification technology with one or more polynucleotides of the invention.

In a further aspect the invention provides vaccines for prevention and/or treatment of SVII infection. The vaccines may be protein-based or DNA-based vaccines. Protein-based vaccines comprise one or more polypeptides derived from SVII, optionally combined with an adjuvant. DNA-based vaccines comprise a isolated polynucleotide encoding a SVII polypeptide or polypeptide fragment operably linked to a promoter active in the subject to be vaccinated (e.g., active in human cells if the subject to be vaccinated is a human).

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987 and periodic updates); “PCR: The Polymerase Chain Reaction”, (Mullis et al., eds., 1994); “Current Protocols in Immunology” (J. E. Coligan et al., eds., 1991 and periodic updates); and “Immunochemistry in Practice” (Johnstone and Thorpe, eds., 1996; Blackwell Science).

Definitions

The terms “Sentinel Virus II” and “SVII” refer to a virus, type of virus, or class of virus which is transmissible via percutaneous exposure in humans and is serologically distinct from hepatitis A virus (HAV), hepatitis B virus (HBV), hepatitis C virus (HCV), hepatitis D virus (HDV), hepatitis E virus (HEV), and hepatitis G virus (HGV). SVII comprises a genome with a major open reading frame (ORF) with at least about 40%, 50%, 55%, 60%, 65%, 70%, 75%, 80%, 85%, 90%, 95%, 97%, 98%, 99% or 100% global amino acid sequence homology with the amino acid sequence of FIG. 1 and/or at least about 40%, 50%, 55%, 60%, 65%, 70%, 75%, 80%, 85%, 90%, 95%, 97%, 98%, 99% or 100% global amino acid sequence identity with the amino acid sequence of FIG. 1. Alternately, a “SVII variant” may have at least about 40%, 50%, 55%, 60%, 65%, 70%, 75%, 80%, 85%, 90%, 95%, 97%, 98%, 99% or 100% global nucleotide sequence identity with the sequence of FIG. 1, encode an ORF with at least about 40%,50%,55%, 60%,65%,70%,75%,80%,85%,90%,95%,97%,98%,99% or 100% global amino acid sequence homology with the amino acid sequence of FIG. 1 and/or at least about 40%, 50%, 55%, 60%, 65%, 70%, 75%, 80%, 85%, 90%, 95%, 97%, 98%, 99% or 100% global amino acid sequence identity with the amino acid sequence of FIG. 1.

A “SVII polypeptide” or “SVII protein” is a polypeptide encoded by an ORF of a SVII virus genome. Exemplary SVII polypeptides are shown in the amino acid sequence shown in FIG. 1. Preferably, a SVII polypeptide is at least about 8, 10, 12, 15, 20, 25, 30, 40, or 50 amino acids and may be less than about 250, 200, 150, 134, 125, 110, 100, 90, 80, 70, 60 or 50 amino acids, wherein the upper and lower limits are independently selected except that the upper limit is always greater than the lower limit.

A “variant SVII polypeptide” is a polypeptide which has at least about 40%, 50%, 55%, 60%, 65%, 70%, 75%, 80%, 85%, 90%, 95%, 97%, 98%, 99% or 100% amino acid sequence homology with the any of the corresponding amino acid sequences of FIG. 1 and/or at least about 40%, 50%, 55%, 60%, 65%, 70%, 75%, 80%, 85%, 90%, 95%, 97%, 98%, 99% or 100% amino acid sequence identity with the corresponding portion of the any of the amino acid sequences of FIG. 1. Preferably, a variant SVII polypeptide is at least about 8, 10, 12, 15, 20, 25, 30, 40, or 50 amino acids and may be less than about 250, 200, 150, 134, 125, 110, 100, 90, 80, 70, 60 or 50 amino acids, wherein the upper and lower limits are independently selected except that the upper limit is always greater than the lower limit.

A “SVII polynucleotide” is a polynucleotide with a sequence identical to a polynucleotide or fragment thereof shown in FIG. 1, a complement thereof, or a polynucleotide which encodes a SVII polypeptide or the complement thereof. Preferably, a SVII polynucleotide is at least about 15, 20, 25, 30, 35, 40, 50 or 60 nucleotides and less than about 371, 350, 300, 250, 200, 150, 125, 75, 50, 40, or 30 nucleotides in length, wherein the upper and lower limits are independently selected except that the upper limit is always greater than the lower limit. A “complement” to a polynucleotide of interest is a polynucleotide having the inverse complement of the reference polynucleotide, according to Watson-Crick base pairing. A complementary polynucleotide is capable of hybridizing under suitable conditions, using Watson-Crick base pairing, to the reference polynucleotide.

A “variant SVII polynucleotide” is a polynucleotide which encodes a variant SVII polypeptide or complement thereof or a polynucleotide which is selectively hybridizable to a SVII polynucleotide or complement thereof, but does not fall within the definition of a SVII polynucleotide. A variant SVII polynucleotide is not found in any known sequence. Preferably, a variant SVII polynucleotide is at least about 15, 20, 25, 30, 35, 40, 50 or 60 nucleotides and less than about 400, 370, 367, 350, 300, 200, 150, 125, 100, 75 or 50 nucleotides in length, wherein the upper and lower limits are independently selected except that the lower limit is always less than the upper limit.

“Amino acid sequence homology” and “amino acid sequence identity” refer to the percentage of amino acids that are homologous or the same in comparing the two sequences. This alignment and the percent sequence homology or sequence identity can be determined using software programs known in the art, for example those described in Current Protocols in Molecular Biology (F. M. Ausubel et al., eds., 1987) Supplement 30, section 7.7.18, Table 7.7.1. Preferably, default parameters are used for alignment. For purposes of the present invention, the alignment program is BLASTP, using the following default parameters: databases=non-redundant (non-redundant GenBank CDS translations+PDB+SwissProt+PIR+PRF), low complexity filtering=ON, expect=10, matrix=BLOSUM62 (gap existence cost 11, gap per residue 1, lambda 0.85) and word size=3. Alignment may be performed gapped or ungapped, and is preferably performed gapped. Details of this BLASTP implementation and these parameters can be found at the following Internet address: http://www.ncbi.nlm.nih.gov/cgi-bin/BLAST.

“Nucleotide sequence identity” refers to the percentage of nucleotide residues which are the same in comparing the two sequences. This alignment and the percent sequence identity can be determined using software programs known in the art, for example those described in Current Protocols in Molecular Biology (F. M. Ausubel et al., eds., 1987) Supplement 30, section 7.7.18, Table 7.7.1. Preferably, default parameters are used for alignment. For purposes of the present invention, the alignment program is BLASTN, using the following default parameters: databases=non-redundant (all non-redundant GenBank+EMBL+DDBJ+PDB sequences), low complexity filtering=ON, expect=10, matrix=BLOSUM62, gap existence cost=5, gap extension cost=2, mismatch penalty=−3, match reward=1, and word size=11. Alignment may be performed gapped or ungapped, and is preferably performed gapped. Details of this BLASTN implementation and these parameters can be found at the following Internet address: http://www.ncbi.nlm.nih.gov/cgi-bin/BLAST.

A polynucleotide which is “selectively hybridizable” to a SVII polynucleotide sequence is one which (i) hybridizes to a SVII polynucleotide sequence without hybridizing to a known virus polynucleotide sequence or specifically primes amplification of a SVII polynucleotide sequence without priming amplification of a known virus polynucleotide sequence. Hybridization of a selectively hybridizable polynucleotide may be accomplished at high stringency, moderate stringency, or low stringency (e.g., allowing for mismatches), as appropriate. High stringency conditions utilize a final wash that is 12-20° C. below the Tm of the expected hybrid, while moderate and low stringency hybridizations utilize final wash conditions which are 21-30° C. and 31-40° below the Tm of the hybrid. The Tm of a long polynucleotides can be found as Tₘ=81.5-16.6(log₁₀[Na⁺])+0.41(% G+C)−0.63(% formamide)−600/N, where N=the length of the selectively hybridizable polynucleotide under study, while the Tₘ of oligonucleotides from about 70 to 15 nucleotides in length may be found as Tₘ=81.5-16.6(log10[Na⁺])+0.41(% G+C)−600/N, and the Tₘ of short oligonucleotides of ≦14 nucleotides may be found as Tₘ=2(A+T)+4(G+C). Priming of amplification is preferably carried out under standard conditions for the polymerase chain reaction (PCR) (e.g., 50 mM KCl, 10 mM Tris-HCl, pH 8.3 (at 20° C.), 1.5 mM MgCl₂, optionally with 0.01% gelatin) and AmpliTaq Gold™ (PE Biosystems) a modified version of T. aquaticus DNA polymerase.

An “isolated” virus, viral structure (e.g., capsid), polynucleotide or polypeptide is one which has been at least partially purified away from contaminating components found in its normal environment. For example, an isolated virus is one which has been at least partially purified away from blood, serum, or tissue proteins. In the case of an isolated viral polynucleotide, the polynucleotide is at least partially purified away from viral proteins and/or other viral components and may additionally be removed from its normal milieu (e.g., nucleotide sequences which normally flank the polynucleotide may be deleted).

As used herein, a sequence of interest and regulatory sequences are said to be “operably linked” when they are covalently linked in such a way as to place the expression or transcription of the sequence of interest under the influence or control of the regulatory sequences. The term “operably linked” relates to the orientation of polynucleotide elements in a functional relationship. Operably linked means that the DNA sequences being linked are generally physically contiguous and, where necessary to join two protein coding regions, contiguous and in the same reading frame. However, since enhancers generally function when separated from the promoter by several kilobases and intronic sequences may be of variable length, some polynucleotide elements may be operably linked but not contiguous. If it is desired that a sequence of interest be translated into a functional protein, two DNA sequences are said to be operably linked if induction of a promoter in the 5′ regulatory sequences results in the transcription of the sequence of interest and if the nature of the linkage between the two DNA sequences does not (1) result in the introduction of a frame-shift mutation, (2) interfere with the ability of the promoter region to direct the transcription of the sequence of interest, or (3) interfere with the ability of the corresponding RNA transcript to be translated into a protein. Thus, a promoter region would be operably linked to a sequence of interest if the promoter region were capable of effecting transcription of that DNA sequence such that the resulting transcript might be translated into the desired protein or polypeptide. It should be noted that the term “operably linked” encompasses operable linkages in which the final protein coding region requires a frameshift or a splice to maintain the proper reading frame through the entire protein coding region (e.g., as seen in certain retroviral systems).

As used herein, the term “antibody” means an immunoglobulin molecule or a fragment of an immunoglobulin molecule having the ability to specifically bind to a particular antigen. Antibodies are well known to those of ordinary skill in the science of immunology. As used herein, the term “antibody” means not only intact antibody molecules but also fragments of antibody molecules retaining antigen binding ability. Such fragments are also well known in the art and are regularly employed both in vitro and in vivo. In particular, as used herein, the term “antibody” means not only intact immunoglobulin molecules of any isotype (IgA, IgG, IgE, IgD, IgM) but also the well-known active (i.e., antigen-binding) fragments F(ab′)2, Fab, Fv, scFv, Fd, V_{H} and V_{L}. For antibody fragments, see, for example “Immunochemistry in Practice” (Johnstone and Thorpe, eds., 1996; Blackwell Science), p. 69. The term “antibody” further includes single chain antibodies, CDR-grafted antibodies, diabodies, chimeric antibodies, humanized antibodies, and a Fab expression library. The term also includes fusion polypeptides comprising an antibody of the invention and another polypeptide or a portion of a polypeptide (a “fusion partner”). Examples of fusion partners include biological response modifiers, lymphokines, cytokines, and cell surface antigens. “Antibody activity” refers to the ability of an antibody to bind a specific antigen in preference to other potential antigens via the antigen combining site located within a variable region of an immunoglobulin. The term “serologically distinct” describes a polypeptide, protein or virus that can be immunologically identified by specific antibodies as distinct from other species of polypeptides, proteins or viruses by virtue of its antigenic differences from such other species.

As used herein, the term “comprising” and its cognates are used in their inclusive sense; that is, equivalent to the term “including” and its corresponding cognates.

Isolated SVII Virus

Isolated SVII is preferably prepared from plasma or serum derived from a SVII infected individual. SVII virus may be isolated from serum or plasma using any technique known in the art, including, but not limited to, sedimentation, isopycnic gradient centrifugation, particularly at preparative scale, and immunoisolation.

SVII viral particles may be isolated using sedimentation techniques (e.g., ultracentrifugation) known in the art. Material containing SVII virus is treated to remove large debris and any cells (e.g., by filtration or medium/high speed centrifugation), then ultracentrifuged to sediment SVII virus. Preferably, the material containing SVII is diluted prior to sedimentation of the virus, such as with tris-buffered saline, or tris-buffered saline with EDTA. For example, SVII virus may be sedimented by centrifugation at 200,000×g for 18 hours (e.g., 200,000×g in a SW41Ti rotor, with the SVII-containing serum diluted with TEN buffer).

Analysis of SVII by isopycnic gradient centrifugation indicates that SVII has a density of ˜1.26 g/cm³ as measured using a sucrose density gradient, and ˜1.26 to 1.28 g/cm³ on a CsCl₂ gradient. Isopycnic gradient centrifugation may be performed using any gradient-forming compound known in the art that will form an appropriate gradient, preferably a gradient forming compound that will form a gradient of approximately 1.2 to 1.35 grams per cubic centimeter (g/cm³);sucrose and cesium chloride (CsCl) are preferred gradient forming compounds. Plasma or serum containing SVII is layered over sucrose gradient or introduced as a homogenous mixture in CsCl in an appropriate centrifuge tube, then centrifuged to equilibrium. Isolated SVII virus may be recovered by collecting the appropriate density fraction of the gradient.

Immunoisolation techniques utilize SVII-specific antibodies in combination with any appropriate separation media known in the art. Preferred separation media include solid plastic substrates (e.g., as for use in panning), chromatographic media (e.g., immunoaffinity chromatography) and magnetic particles (e.g., immunomagnetic separation). The SVII antibodies are conjugated to the separation media, which is exposed to a material containing SVII virus. Unbound materials are removed and residual unbound materials are washed away from the immunoseparation substrate, then the bound SVII is eluted, typically by use of an elution buffer with altered pH or high salt concentration.

Alternately, isolated SVII virus may be prepared by in vitro culture methods. A variety of such methods are known in the art, and normally comprise infecting an appropriate host cell, preferably a liver cell line, with SVII, culturing the infected cells, and collecting SVII viral particles from the culture media or by lysing the host cells. Density gradient separation or immunoisolation techniques may be used to further isolate the virus.

Isolated SVII polynucleotides

Isolated SVII polynucleotides may be prepared by any method known in the art, such as by direct isolation of viral DNA from viral particles, by direct isolation of viral RNA transcribed as part of the SVII life cycle, by use of a hybridization method (i.e., identification of viral DNA in DNA libraries prepared from viral DNA, or virus-containing serum or plasma), by use of an amplification method (i.e., polymerase chain reaction of viral DNA, viral-DNA containing libraries, or DNA isolated from plasma or serum), or by direct synthesis. The polynucleotide sequence shown in FIG. 1 may be used to design probes or primers for use in hybridization and amplification methods and to select sequences for synthesis. Preferably, the selected probes or primers are unique (e.g., are not found in GenBank or other sequence databases).

Isolated genomic polynucleotides may be prepared by extraction of isolated viral particles. Isolated viral particles may be subjected to any DNA extraction technique known in the art, such as guanidinium HCl extraction, optionally followed by further purification and/or concentration techniques such as agarose gel purification, phenol/chloroform extraction, or ethanol precipitation in the presence of salts.

Preparation of DNA libraries is well known in the art. DNA isolated from viral particles or virus-containing plasma or serum may be cloned into a convenient library vector using techniques commonly used in the art. Most commonly, the library will be prepared using a lambda phage-based library vector, although cosmid and plasmid libraries are also commonly used. Phage-based libraries are plated by infection of ‘lawns’ of E. coli host cells, while cosmid and plasmid libraries are typically transformed into cells which are plated. After plating, DNA from the library is transferred to screening filters, and screening with a SVII polynucleotide probe. The probe is preferably modified such that hybridization can be detected, typically by the incorporation of a radioactive nucleotide (e.g., ³²P), although other modified probes (e.g., digoxigenin or biotin labeled) may be detected through the use of a modified enzyme (e.g., alkaline phosphatase or luciferase) which binds the labeled probe and acts on a chromogenic or otherwise detectable substrate. Clones hybridizing to the SVII polynucleotide probe are purified by one or more ‘rounds’ of purification (e.g., repeating the plating and screening process on progressively more purified clones), as is well known in the art. SVII DNA may be prepared by harvesting DNA from clones isolated in the screening procedure, and optionally further isolated from the library vector DNA by restriction endonuclease digestion. Alternatively, clone DNA isolated by screening may be used as a substrate for amplification of SVII virus DNA using polymerase chain reaction (PCR) methodology. PCR primers may be designed from SVII virus DNA or, more conveniently, may be designed to hybridize to DNA sequences in the library vector which flank the site at which the library DNA was inserted, as will be apparent to one of skill in the art.

SVII virus polynucleotides may also be isolated by amplification from samples containing SVII DNA. Primers for amplification may be designed based on the sequence shown in FIG. 1, and are preferably designed so as to amplify SVII DNA, but not viral DNA from other viruses or genomic DNA from animals or prokaryotes. Additionally, as is well known in the art, the primer sequences are selected to minimize any intramolecular secondary structure, which substantially inhibits, and may even block, amplification. Protocols for polymerase chain reaction amplification are well known in the art, as are protocols for other amplification methods such as ligase chain reaction. After amplification, the SVII DNA may be further purified by size selection (e.g., gel electrophoresis) or chemical extraction (e.g., phenol/chloroform extraction) and/or concentrated by ethanol precipitation in the presence of salts.

SVII polynucleotides may also be chemically synthesized, although synthesized SVII polynucleotides are preferably less than about 50-60 nucleotides in length, as yields for polynucleotide synthesis drop as chain length increases. Methods for synthesis of polynucleotides are well known in the art, and generally involve the iterative addition of nucleotides (or modified nucleotides) to the growing end of the synthetic polynucleotide. A variety of different systems are available in the art, and the selection of the particular method and chemistry is left to the practitioner.

SVII polynucleotides have a variety of uses, including detection of SVII virus (which is useful in diagnosis of SVII infection), production of SVII polypeptides, construction of SVII-based expression/transduction vectors, and as antisense oligonucleotides or for construction of antisense SVII vectors.

Antisense SVII polynucleotides are SVII polynucleotides which are capable of selective hybridization to a segment of an mRNA molecules produced from an SVII genome. Antisense SVII polynucleotides may be any size SVII polynucleotide, but are preferably less than about 200 nucleotides length. Antisense SVII polynucleotides block expression of SVII proteins and/or SVII viral replication in SVII infected cells. Accordingly, SVII antisense polynucleotides may be used to treat SVII infections and/or ameliorate the symptoms of SVII infections, including reduction of SVII viremia.

When SVII antisense polynucleotides are chemically synthesized, they are preferably synthesized as modified oligonucleotides to increase resistance to nucleases. Modified oligonucleotides may be synthesized to include phosphoroamidites at the 5′ and 3′ termini (Dagle et al., 1990, Nucl. Acids Res. 18:4751-4757), to incorporate the ethyl or methyl phosphonate analogs disclosed in U.S. Pat. No. 4,469,863, to incorporate phosphorothioate (Stein et al., 1988. Nucl. Acids Res. 16:3209-3221) or 2′-O-methylribonucleotides (Inove et al., 1987, Nucl. Acids Res. 15:6131), or as chimeric oligonucleotides that are composite RNA-DNA analogues (Inove et al., 1987, FEBS Lett. 215:327).

SVII antisense polynucleotides may be delivered to individuals infected with SVII virus as “naked DNA,” normally by parenteral injection, preferably by intravenous or introduction into the portal vein, exploiting the naturally occurring uptake of oligonucleotides. Alternately, SVII antisense polynucleotides may be introduced into target cells via a vector, such as a viral vector. The vector comprises a promoter operable in a host cell, preferably a human host cell infected with SVII, operably linked to a polynucleotide sequence, transcription of which results in the production of an SVII antisense polynucleotide. Preferred viral vectors include, but are not limited to, the adeno-associated viral vectors known in the art. Preferably, SVII antisense polynucleotides delivered by viral vector are administered intravenously, preferably into the portal vein.

Isolated SVII Polypeptides

SVII proteins may comprise an entire ORF from a SVII virus, one or more fused proteins from a SVII virus, a single protein from a SVII virus, or fragments thereof. Also included are “mosaic proteins” which comprise two or more SVII protein fragments within the same protein. The SVII protein fragments in a mosaic protein may be from the same SVII protein or from different SVII proteins. Where the SVII protein fragments are from the same SVII protein, the amino acid sequence normally separating the fragments is substantially deleted or replaced with an unrelated “spacer” sequence. Another mosaic protein encompassed by the invention is a “superepitope” mosaic protein that comprises homologous versions of at least one epitope from at least two different SVII viruses. Superepitope mosaic proteins may be used, for example, in screening assays to generically detect SVII virus infection.

SVII polypeptides may be prepared by any method known in the art, including purification from isolated viral particles, recombinant production and chemical synthesis. Due to the relative difficulty of isolating large amounts of viral particles from natural sources, recombinant production and/or chemical synthesis are preferred methods for production.

Recombinant production of proteins is well known in the art. Generally, a polynucleotide sequence encoding a protein of the invention is cloned into an “expression vector,” which is introduced into a suitable host cell. The host cell is cultured under conditions appropriate for expression of the protein, and the recombinant protein is collected. The exact details of the expression construct will, as will be apparent to one of skill in the art, vary depending on the desired host cell and properties of the expression construct, although the expression construct will normally include a promoter/operator or promoter/enhancer operable in the host cell and a selectable marker allowing selection of cells containing the marker. Preferably the promoter/operator or promoter/enhancer is ‘controllable’ in that a change in culture conditions will lead to expression of the SVII protein (or SVII protein fusion protein).

It should be noted that SVII peptides may be incorporated into “fusion proteins” for recombinant production. A fusion protein comprises a protein of interest (e.g., the SVII protein) linked to a fusion partner, and optionally includes a specific cleavage site between the protein of interest and the fusion partner to allow separation of the two parts. The fusion partner may be at the amino terminal or carboxy terminal of the protein, although fusion proteins which incorporate the protein of interest as an ‘insert’ within the sequence of the coding region, are also contemplated. Fusion proteins comprising a SVII protein insert may be particularly useful as screening tools, for example, when incorporated into a “phage display” system (e.g., where the SVII protein sequence is inserted into a lambda phage coat protein).

Useful fusion partners include proteins which allow for easy purification of the fusion protein (e.g., glutathione-S-transferase, oligo-histidine, and certain sequences derived from the myc oncogene), increase solubility of the fusion protein (such as E. coli DsbA, disclosed in U.S. Pat. No. 5,629,172), or create a “linker” to bind the protein to a substrate (e.g., polyglycine with a terminal lysine could be used to link a SVII protein to substrate for use in an immunoassay).

Generally, an expression construct is created by inserting a polynucleotide encoding a protein of the invention into an appropriate recombinant DNA expression vector using appropriate restriction endonucleases. The restriction endonuclease sites may be naturally occurring or synthetic sites that have been introduced by any method known in the art, such as site-directed mutagenesis, PCR or ligation of linker/adapters to the polynucleotide. Alternately, the polynucleotide may be a synthetic sequence, designed to incorporate convenient restriction enzyme sites and/or optimize codon usage for the intended host cell. The particular endonucleases employed will be dictated by the restriction endonuclease cleavage pattern of the parent expression vector to be employed. The choice of restriction sites is made so as to properly orient the coding sequence with control sequences to achieve proper in-frame reading and expression of the protein.

The polynucleotide may be inserted into any appropriate expression vector. Expression vectors may be found in a number of forms, including, but not limited to, plasmid, cosmid, yeast artificial chromosome (YAC), and viral. In general, the expression vector will contain an autonomous replication site that is active at least in the organism in which the vector is propagated, and frequently also in the recombinant host cell. The expression vector will also typically include marker sequences which are capable of providing phenotypic selection in transformed cells, such as positive selection markers, such as an antibiotic resistance genes (e.g., bla, tet^{R}, neo^{R} or hyg^{R}) or genes which complement an auxotrophy (e.g., trp or DHFR) and/or negative selection markers such as herpes simplex virus 1 thymidine kinase. The expression vector will also include necessary sequences for initiation and termination of transcription and translation (e.g., promoter, Shine-Dalgarno sequence, ribosome binding site, transcription termination site) and may optionally contain sequences which modulate transcription (e.g., SV40 enhancer or lac repressor), and may also contain sequences which direct processing, such as an intron or a polyadenylation site, as necessary.

The polynucleotide of the invention is inserted into the expression vector in the proper orientation and relationship with the expression vector's transcriptional and translational control sequences to allow transcription from the promoter and translation from the ribosome binding site, both of which should be functional in the host cell in which the protein is to be expressed. The transcriptional control sequences are preferably inducible (i.e., can be modulated by altering the culture conditions, such as the lac operon for E. coli or the metallothionein promoter for mammalian cells). An example of such an expression vector is a plasmid described in Belagaje et al., U.S. Pat. No. 5,304,493. The gene encoding A-C-B proinsulin described in the reference can be removed from the plasmid pRB182 with restriction enzymes NdeI and BamHI. The genes encoding the protein of the present invention can be inserted into the plasmid backbone on a NdeI/BamHI restriction fragment cassette.

Microbial hosts are normally preferred for recombinant expression of the proteins of the invention, and any commonly used microbial host, including E. coli such as W3110 (prototrophic, ATCC NO. 27325), Bacillus subtilis, and other enterobacteriaceae such as Salmonella typhimurium or Serratia marcescans, and various pseudomonas species may be used. Alternately, eukaryotic host cells, including yeast such as Saccharomyces cerevisiae, Schizosaccharomyces pombe, as well as higher eukaryotes such as non-yeast fungal cells, plant cells, insect cells (e.g., Sf9), and mammalian cells (e.g., COS, CHO) may be used.

The completed expression construct is introduced into the recombinant host cell by any appropriate method known in the art, such as CaCl₂ transfection, Ca₂PO₄ transfection, viral transduction, lipid-mediated transfection, electroporation, ballistic transfection, and the like. After introduction of the expression construct, the recombinant host cell is generally cultured under appropriate conditions to select for the presence of the expression construct (e.g., cultured in the presence of ampicillin for a bacterial host with an expression construct containing bla), or alternately may be selected for expression of the protein by any appropriate means (e.g., fluorescence activated cell sorting, FACS, using a SVII protein-specific antibody).

After selection and appropriate isolation procedures (e.g., restreaking or limiting dilution cloning), the recombinant host cells are cultured at production scale (which may be from 500 mL shaken flask to multi-hundred liter fermenter for microbial host cells, or from T25 flask up to multi-hundred liter bioreactor for mammalian host cells, depending on the requirements of the practitioner), using any appropriate technology known in the art. If the promoter/enhancer in the expression vector is inducible, the expression of the protein is induced as appropriate for the particular construct (e.g., by adding an inducer, or by allowing a repressor to be depleted from the media) after the culture reaches an appropriate cell density, otherwise the cells are grown until they reach appropriate density for harvest. Harvesting of the recombinant proteins of the invention will depend on the exact nature of the recombinant host cells, the expression construct, and the polynucleotide encoding the protein of the invention, as will be apparent to one of skill in the art. For expression constructs that result in a secreted protein, the protein is normally recovered by removing media from the culture vessel, while expression constructs that result in intracellular accumulation of the protein generally require recovery and lysis of the cells to free the expressed protein.

Proteins which are expressed in high-level bacterial expression systems characteristically aggregate in granules or inclusion bodies which contain high levels of the overexpressed protein. The protein aggregates are solubilized to provide further purification and isolation of the desired protein product, for example, using strongly denaturing solutions such as guanidinium-HCl, possibly in combination with a reducing agent such as dithiothreitol (DTT). The solubilized protein is recovered in its active form after a “refolding” reaction, in which generally involves reducing the concentration of the denaturant and adding oxidizing agent. Protocols which are considered generally applicable for the refolding of proteins are well known in the art, and are disclosed in, for example, U.S. Pat. Nos. 4,511,502, 4,511,503, and 4,512,922.

Short (e.g., less than about 20 amino acid residues) SVII proteins may also be conveniently produced using synthetic chemistry, a process well known in the art. Due to decreased yields at long peptide lengths, synthesis is a preferred method for production of peptides of about 15 amino acid residues or less.

SVII polypeptides may be used in vaccines for prevention of SVII infection and/or treatment of SVII infection. Any SVII polypeptide or combination of SVII polypeptides maybe used in a SVII vaccine. SVII mosaic polypeptides comprising multiple epitopes from a single SVII protein, wherein the amino acids normally separating the epitopes are deleted are one preferred SVII protein for use in a vaccine formulation. Another preferred SVII protein for use in a vaccine is a superepitope protein which comprises homologous epitopes from multiple SVII viruses fused into a single protein.

SVII vaccines are formulated according to methods known in the art. Preferably the vaccine is in a liquid formulation for parenteral administration. The vaccines may be formulated including pharmaceutical excipients known in the art such as physiologically and pharmaceutically acceptable salts, buffers, preservatives, bulking agents, osmolyte agents, and the like, which may be found in the USP (UNITED STATES PHARMACOPEIA, United States Pharmacopeial Convention, Inc., Rockville, Md., 1995).

SVII protein-based vaccines may also be formulated with adjuvants. Adjuvants for use in SVII protein-based vaccines include chemical adjuvants such as aluminum hydroxide (especially aluminum hydroxide gels), potassium alum, protamine, aluminum phosphate and calcium phosphate, cytokine adjuvants including interleukin 1.beta, tumor necrosis factor alpha, and granulocyte-macrophage colony stimulating factor (GM-CSF), such as described in U.S. Pat. No. 5,980,911, and oil in water emulsions such as Freund's complete and incomplete adjuvants.

SVII vaccines are preferably delivered parenterally, more preferably by percutaneous administration. Prefered routes of administration include intramuscular and subcutaneous injection as well as percutaneous air-driven administration (e.g., needless injection). The vaccine may be given in a single dose or as multiple administrations. Where multiple administrations are given, they are preferably separated by at least one day, week, or month.

SVII Antibodies

Antibodies against SVII may be prepared using the isolated viral particles and/or SVII viral proteins provided by the instant invention. Isolated polyclonal antibodies as well as monoclonal antibodies may be made.

Isolated polyclonal antibodies against SVII proteins are preferably prepared by injecting a “SVII immunogen” (e.g., isolated SVII viral particles, SVII protein(s), SVII oligopeptides linked to a carrier, or SVII fusion proteins) in an immunogenic form into an animal, preferably a mammal such as a rodent (e.g., a mouse, rat or rabbit), a goat, a cow or a horse. Most commonly, the first injection of SVII immunogen is made as an oil/water emulsion complete adjuvant such as Freund's complete adjuvant, which contains a non-specific activator of the immune system to improve immune response to the injected immunogen. Later injections are typically made with incomplete adjuvant (e.g., in an emulsion w/o a non-specific immune stimulator). Alternately, the SVII immunogen can be introduced adsorbed to a solid substrate or as a simple solution. Serum is harvested and tested for the presence of specific antibody using any convenient assay, most typically a simple immunoassay such as an ELISA (enzyme-linked immunosorbent assay) using a SVII immunogen as the target and a species-specific anti-immunoglobin secondary antibody.

Monoclonal antibodies of this invention can be prepared by a number of different techniques. For hybridoma technology, the reader is referred generally to Harrow & Lane (1988), U.S. Pat. Nos. 4,491,632, 4,472,500, and 4,444,887, and Methods in Enzymology, 73B:3 (1981). Traditional monoclonal antibody technology involves the immortalization and cloning of an antibody-producing cell recovered from an animal, typically a mouse, that has been immunized as described in the preceding paragraph. The cell may be immortalized by, for example, fusion with a non-producing myeloma, infection with Epstein Barr Virus, or transformation with oncogenic DNA. The treated cells are cloned and cultured, and clones are selected that produce antibody of the desired specificity. Specificity testing is performed on culture supernatants by a number of techniques, such as using the immunizing antigen as the detecting reagent in an immunoassay. A supply of monoclonal antibody from the selected clone can then be purified from a large volume of culture supernatant, or from the ascites fluid of suitably prepared host animals injected with the clone.

Alternative methods for obtaining monoclonal antibodies involve contacting an immunocompetent cell or viral particle with a protein of the invention. In this context, “immunocompetent” means that the cell or particle has expressed or is capable of expressing an antibody specific for the antigen without further genetic rearrangement, and can be selected from a cell mixture by presentation of the antigen. Immunocompetent eukaryotic cells can be harvested from an immunized mammalian donor, or they can be harvested from an unimmunized donor and prestimulated in vitro by culturing in the presence of immunogen and immunostimulatory growth factors. Cells of the desired specificity can be selected by contacting with the immunogen under culture conditions that result in proliferation of specific clones but not non-specific clones. Immunocompetent phage may be constructed to express immunoglobulin variable region segments on their surface. See Marks et al., New Engl. J. Med. 335:730, 1996; International Patent Applications Nos. 94/13804, 92/01047, 90/02809; and McGuinness et al., Nature Biotechnol. 14:1149, 1996. Phage of the desired specificity may be selected, for example, by adherence to a SVII immunogen attached to a solid phase, and then amplified in E. coli.

Antibody can be purified from serum, cell supernatants, lysates, or ascites fluid by a combination of traditional biochemical separation techniques, such as ammonium sulfate precipitation, ion exchange chromatography on a weak anion exchange resin such as DEAE, hydroxyapatite chromatography, and gel filtration chromatography. Specific affinity techniques, such as affinity chromatography using a SVII immunogen as the affinity moiety may also be used, alone or in conjunction with traditional biochemical separation techniques to isolate antibodies of the invention.

Antibodies obtained are preferably screened or purified not only for their ability to react with SVII viral proteins, but also for a low cross-reactivity with potential cross-reacting substances also present in samples of diagnostic interest. Unwanted activity can be adsorbed out of polyclonal antisera, if necessary, using the cross-reacting substance or an antigen preparation from serum from an individual negative for SVII infection.

The epitope to which a particular antibody binds can be mapped by preparing fragments and testing the ability of the antibody to bind. For example, sequential peptides of 12 amino acids are prepared covering the entire sequence of the immunogen, and overlapping by 8 residues. The peptides can be prepared on a nylon membrane support by F-Moc chemistry, using a SPOTS™ kit from Genosys according to manufacturer's directions. Prepared membranes are then overlaid with the antibody, washed, and overlaid with anti-human IgG conjugated with either alkaline phosphatase or horseradish peroxidase. The test is developed by adding the appropriate substrate for the particular enzyme conjugate used. Positive staining indicates an antigen fragment recognized by the antibody. The fragment can then be used to obtain other antibodies recognizing the epitope of interest. Two antibodies recognizing the same epitope will compete for binding in a standard immunoassay.

Antibodies of the invention may be used for the detection and/or identification of SVII virus, and may also be useful in isolation of viral particles and/or viral proteins.

Detection of SVII

The polynucleotides, proteins, and antibodies of the invention may be used in methods and kits for the detection of SVII viral infection and detection of SVII itself. Assays using the polynucleotides, proteins, and/or antibodies of the invention may be designed in a variety of formats, depending on the desired utility of the assay.

Polynucleotides of the invention may be used for detection of SVII virus genomic DNA. Detection of SVII genomic DNA in blood samples indicates that the sample is contaminated with SVII virus and that the source of the sample is infected with SVII. A wide variety of different assays for detection of nucleotides are known, although all such assays generally require a hybridization step where a primer or probe is hybridized to DNA in the sample.

Using determined portions of the isolated SVII genomic sequence as a basis, oligomers of approximately eight nucleotides or more can be prepared, either by excision or synthetically, which hybridize with the SVII genome. The natural or derived probes for SVII polynucleotides are a length which allows the detection of unique viral sequences by hybridization. Generally, probes are a minimum of six to eight nucleotides in length, sequences of at least ten to twelve nucleotides are preferred, and those of at least about 20 nucleotides may be most preferred. Depending on the desired utility of the assay (e.g., detection of all SVII viruses vs. detection of a single SVII virus type), the probe may be based on a region of SVII genomic sequence which is conserved among SVII viruses or highly divergent among SVII viruses. These probes can be prepared using routine, standard methods including automated oligonucleotide synthetic methods. A complement of any unique portion of the SVII genome will normally be satisfactory. Generally, complete complementarity is desirable in probes, although it may be unnecessary as the length of the fragment is increased.

Normally, the test sample to be analyzed, such as blood or serum, is treated such as to extract the nucleic acids contained therein. The nucleic acid sample is typically adsorbed to a solid support (e.g., nitrocellulose) for the assay (with or without preliminary size separation such as by gel electrophoresis), although solution phase format assays such as the assay described in U.S. Pat. No. 4,868,105 may also be used.

Depending on the format of the assay and the detection system, the probes may or may not be directly labeled or otherwise modified to allow later detection by binding of a label. Suitable labels and methods for attaching labels to probes are known in the art, and include but are not limited to radioactive labels incorporated by nick translation or kinasing, modifications which allow later binding of a label, such as biotinylation, as well as fluorescent and chemiluminescent labels which may be directly bound to the probe or bound via a modification of the probe.

In the basic nucleic acid hybridization assay, single-stranded sample nucleic acid is contacted with the probe under hybridization and wash conditions of suitable stringency, and resulting duplexes are detected. Control of stringency is well known in the art, and depends on variables such as salt concentration, probe length, formamide concentration, temperature and the like. Preferably, hybridization and washing is performed under stringent conditions. Detection of bound probe is performed according to requirements of the label/detection system employed in the assay. For example, where the probe is radioactively labeled, probe binding is detected by autoradiography. Where the probe is modified to allow later binding of a label (e.g., by covalent linkage of biotin or digoxigenin to the probe or by addition of a polyA tail to the probe), a label linked to a modification binding moiety (e.g., streptavidin linked to a detectable enzyme such as alkaline phosphatase, green fluorescent protein or luciferase, or a fluorescent or other label bound to an anti-digoxigenin antibody). Detection of fluorescent probes is generally accomplished by fluorimeter, while luminescent labels may be detected using a luminometer or a photographic plate. Branched DNA technology and other methods which amplify signal from the assay may be employed (Urdea et al., 1989, Clin. Chem. 35(8):1571-1575; U.S. Pat. No. 5,849,481;).

Other assays employ probes as primers for amplification of SVII genomic DNA in the sample. Methods such as polymerase chain reaction, ligase chain reaction, Q-beta replicase, NASBA (Compton, 1991, Nature 350(6313):91-92), etc., maybe used to create large numbers of copies of a portion or all of the SVII genomic DNA present in a sample. Detection in such assays is normally by detection of an amplification product of an expected size, typically by gel electrophoresis and visualization of any bands present.

SVII virus may also be detected using antibodies of the invention to detect the presence of viral proteins in a sample. Any of the wide variety of immunoassay formats known in the art may be used in conjunction with antibodies of the invention for detection of SVII virus or viral proteins.

In its most basic form, an immunoassay for detection of SVII virus or SVII protein in a sample detects a complex of SVII protein(s) with an antibody of the invention. At least one antibody of the invention is required, although preferred immunoassay formats require at least two antibodies of the invention.

Many assay formats require that sample or the SVII proteins from the sample, be immobilized a solid support. Linkage can be accomplished by a variety of means known in the art, most commonly adsorption to a protein binding surface (e.g., a polystyrene plate or nitrocellulose or PVA membrane) or binding to an antibody which is bound to the substrate. This second arrangement is used in “sandwich” immunoassays and is preferred for detection of SVII virus proteins.

After immobilization of the sample (or the SVII proteins in the sample) to the substrate, a detection antibody is contacted with the sample and the presence of the detection antibody is detected. The detection antibody may itself be detectable due to modification of the antibody with a dye or colored particles, it may be modified such that a detection reagent will bind to the detection antibody, or it may be modified with an enzyme which acts on a chromogenic substrate The exact details of detection of the detection antibody will, of course, depend on the detection system utilized. Directly detectable detection antibodies may be detected by, for example, simple inspection, light microscopy or colorimetry (for antibodies modified with colored particles such as latex beads or colloidal metals), radiometry (for antibodies modified with a radioactive compound) or fluorimetry or epifluorescence microscopy (for antibodies labeled with fluorescent dyes). Detection antibodies which have been modified to include an enzyme are typically detected by incubating the assay in a solution containing a substrate which becomes detectable upon processing by the enzyme (e.g., substrates which change color or become fluorescent or luminescent after processing by the enzyme) and detecting any processed substrate using an appropriate method (e.g., colorimetry for chromogenic substrates, fluorimetry for fluorescent substrates, and the like). Other detection antibodies may be modified to allow for “indirect” detection, where a second reagent which binds to the modified detection antibody allows detection of bound detection antibody. The second reagent is modified such that it is detectable (either directly as with a dye or colored particle, indirectly as with an enzyme and detectable substrate).

### EXAMPLES

### Example 1

### Isolation of SVII Virus DNA

DNA clones comprising SVII genomic DNA were isolated using a modification of the representation difference analysis (RDA) method described by Lititsyn et al. (1993, Science 259:946-951). This method utilizes a “driver” DNA source to enrich amplification of sequences unique to the “tester” DNA source.

Serum from a cryptogenic hepatitis patient designated H101 was used as the source of “tester” DNA. DNA was extracted by proteinase K digestion followed by phenol and chloroform extraction. DNA isolated from 100 μL H101 serum was digested to completion with 10 units of Sau3A I for three hours at 37° C. The enzyme was inactivated by incubation at 65° C. for 20 minutes.

Linkers R-Bgl-24 (5′-AGCACTCTCCAGCCTCTCACCGCA-3′) and R-Bgl-12 (5′-GATCTGCGGTGA-3′) were ligated to the digested DNA by mixing the digested DNA with 1 nmol of each oligo in T4 DNA ligase buffer (with ATP, from New England Biolabs), denaturing the mixture by incubation for two minutes at 55° C., annealing the linkers by gradually cooling the mixture to 10-15° C. over about an hour, then adding 800 units of T4 DNA ligase (New England Biolabs) and incubating overnight at 12-16° C.

Tester amplicons were prepared by nested PCR reactions, since one round of PCR did not yield quantifiable amount of DNA. A portion of the ligation product was mixed with PCR buffer, dNTPs, and an additional 250 pmol of R-Bgl-24 oligo and overlaid with mineral oil. The R-Bgl-12 oligo was ‘released’ by incubating the mixture for three minutes at 72° C. Overhangs were filled by adding 7.5 units of AMPLITAQ® Taq DNA polymerase (PE Biosystems) and incubating for a further five minutes at 72° C. Tester amplicons were created by running the mixture through 20 cycles of one minute at 95° C. and three minutes at 72° C. followed by a final extension step at 72° C. for ten minutes. 10 μl of first round PCR product and linker R-Bgl-25 (5′-ACTCTCCAGCCTCTCACCGCAGATC-3′) were used in the second round of PCR reaction under the same conditions as the first round for 15 cycles. The product was then extracted with phenol/chloroform and precipitated with sodium acetate and isopropanol. The precipitate was collected by centrifugation, air dried after removal of the supernatant, and resuspended in TE (tris-EDTA) buffer. The R-Bgl-24 linkers were removed by Sau3A I digestion essentially as above, followed by inactivation of the enzyme at 65° C. The digestion product was precipitated using sodium acetate and ethanol in the presence of glycogen, collected by centrifugation, air dried following removal of the supernatant, and resuspended in TE. The product was then separated on a 1% agarose gel run in lx TAE, and the portion of the gel corresponding to 150-1500 nucleotides was cut out. Digested tester amplicons were purified from the gel using a QIAGEN® Qiaex II Gel Extraction kit according to the manufacturer's instructions. 2 μg of tester amplicon DNA was ligated with J-Bgl-24 and J-Bgl-12 linkers (5′-ACCGACGTCGACTATCCATGAACA-3′ and 5′-GATCTGTTCATG-3′, respectively), essentially as described for the R-Bgl linkers.

Driver amplicons were prepared from DNA extracted from serum pooled from 10 healthy blood donors essentially as described for tester amplicons, except that new linkers were not added after the second Sau3A I digestion.

Driver and tester amplicons were mixed at a 100:1 mass ratio, extracted with phenol/chloroform, and precipitated with sodium acetate and ethanol. The pellet was collected by centrifugation, air dried following removal of the supernatant, and resuspended in 4 μL of EE×3 buffer (30 mM EPPS, pH 8.0, 3 mM EDTA). The mixture was overlaid with mineral oil hybridized by denaturing for five minutes at 98° C., adding 1 μL of 5 M NaCl, incubating a further two minutes at 98° C., then 20 hours at 65° C.

The tester/driver hybridization mix was amplified under conditions which selectively amplify only double stranded tester DNA. A portion of the hybridization mix was amplified for 10 cycles essentially as was done for amplification of the J-Bgl ligated tester DNA, except that the extension cycles were performed at 70° C. The amplification product was collected, extracted with phenol/chloroform/isoamyl alcohol, then precipitated with sodium acetate and isopropanol. The precipitate was collected by centrifugation, air dried following removal of the supernatant, and resuspended in TE. Single stranded DNA was removed by digestion with mung bean nuclease (New England BioLabs) for 30 minutes at 30° C., followed by heat inactivation of the enzyme at 98° C. for five minutes. The digestion product was the re-amplified for 15 cycles in the presence of additional J-Bgl-24 oligonucleotide.

The product of the amplification was collected, extracted with phenol/chloroform/isoamyl alcohol, precipitated with sodium acetate and isopropanol, collected by centrifugation, washed with 70% ethanol, air dried following removal of the supernatant, and resuspended in TE to form the First Difference Product (DPI).

The Second Difference Product (DP2) was created by digesting DPI with Sau3A I and substituting N-Bgl linkers (N-Bgl-12 and N-Bgl-24, 5′-GATCTTCCCTCG-3′ and 5′-AGGCAACTGTGCTATCCGAGGGAA-3′, respectively) for the J-Bgl linkers essentially as described for the switch from the R-Bgl linkers to the J-Bgl linkers, hybridizing the N-linker DPI with driver amplicons at a 1:800 mass ratio, and amplifying/digesting/amplifying as described for DP 1, except that extensions during the amplifications were carried out at 72° C.

The Third Difference Product (DP3) was created by digesting DP2 with Sau3A I and substituting J-Bgl linkers for the N-Bgl linkers, followed by followed by hybridization with driver amplicons at a 4×10⁵:1 driver:tester mass ratio, and amplification/digestion/amplification as for DP1.

After three rounds subtractive hybridization and selective amplification, distinct bands were seen after gel electrophoresis, as compared to the ‘smear’ patterns of the original tester amplicons. DNA was isolated from each band using a QIAGEN® Gel Extraction Kit (Cat. No. 28704) according to the manufacturer's instructions, then ligated into TA plasmid 2.1 (InVitrogen Cat. K2000-01). The resulting libraries of plasmids were then transformed into E. coli and plated. 30 colonies from each library were selected and sequenced using a PE Biosciences PCR sequencing kit according to the manufacturer's instructions. Sequences were compared against the GenBank database at the DNA and protein levels. Clones which showed no significant homology with the databases were classified as “unknowns”. Each “unknown” was tested for its presence in human genomic DNA by PCR using primers were designed from each “unknown” sequence. Analysis was discontinued for any sequence present in human genomic DNA. One 371 nucleotide unknown, originally designated “clone 33” or “H101.c33” was selected for further characterization. The nucleotide sequence of H101.c33 is shown in FIG. 1.

The nucleotide sequence of clone 33 was analyzed by conceptual translation in all six possible reading frames. One large open reading frame (ORF) was identified: ORF 1. The amino acid sequence of ORF 1 is also shown in FIG. 1.

The non-human origin of clone 33 was confirmed by PCR. No amplification product was detected after amplification of human genomic DNA using PCR primers specific for clone 33. Upon confirmation of the non-human origin of clone 33, it was redesignated Sentinel Virus II, or SVII.

The presence of SVII was confirmed in patient H101's serum at two time points corresponding to peak ALT levels, using PCR primers specific for SVII.

### Example 2

### Physical Characterization of SVII Virus Particles

SVII positive serum was fractionated by density gradient ultracentrifugation to determine the buoyant density of SVII viral particles.

A 500 μL sample of SVII positive serum, spiked with HBV as a marker, was layered on the surface of a continuous sucrose density gradient (20-65% sucrose, w/w). The sample was centrifuged for 39,000 rpm for 15 hours at 6° C. in a Beckman SW41Ti rotor. Fractions (500 μL) were collected by pumping from the bottom of the tube via glass capillary tube attached to silicone tubing.

Each fraction was assayed for SVII using nested PCR. The first round used primer pair 33.1/33.2 (5′-GGATTGACGACGACGACGAC-3′ and 5′-TGTCAAATACCCGCTCAGGA-3′, respectively), and the second round used primer pair 33.3/33.4 (5′-GACGACGACGACGACATTG-3′ and 5′-CAAATACCCGCTCAGGAAGG-3′, respectively). Fractions were also assayed for HBV using two rounds of PCR; the first round with primers HBV1 and HBV4 (5′-CATCTTCTTRTTGGTCTTCTGG-3′ and 5′-CAAGGCAGGATAGCCACATTGTG-3′, respectively), and primers HBV3 (5′-CCTATGGGAGTGGGCCTCAG-3′) and HBV4. SVII virus was found in fractions corresponding to 1.26 g/cm³.

SVII buoyant density was also measured in CsCl gradient. A 500 μL sample of SVII positive serum, spiked with HBV as a marker, was mixed with a homogenous CsCl solution (density 4.2 g/cm³ and refractive index of 1.3645) in an appropriate centrifuge tube. The sample was centrifuged for 35,000 rpm for 70 hours at 6° C. in a Beckman SW41Ti rotor. Fractions were collected by puncturing the side wall near the bottom of the tube and collecting 500 μL fractions and analyzed as described for the sucrose gradient experiment. SVII was found in fractions corresponding to 1.26-1.28 g/cm³.

### Example 3

### Prevalence of SVII Infection

More than 700 serum samples were assayed by PCR for presence of SVII. The samples were divided into: (a) “super normal” blood donors (normal blood values, no hepatitis virus markers, and not implicated in transfusion-related events for ≧5 blood donations); (b) “normal” blood donors (meeting blood donation criteria); (c) “disqualified” blood donors (healthy individuals not eligible for blood donation under current rules); and (d) “hepatitis” patients, separated into acute hepatitis, chronic HBV, chronic HCV, cryptogenic (nonA-E), and superinfected hepatits (HBV and HCV or HCV and HDV).

Samples were assayed by nested PCR amplification of DNA extracted from serum samples using primer pairs 33.1/33.2 and 33.3/33.4. SVII genomic DNA was not detected in serum samples from individuals meeting blood donation screening criteria, and was found only at a very low prevalence in healthy individuals not meeting blood donation screening criteria. However, SVII was found at a high prevalence in serum from acute hepatitis patients, and was also found in serum samples from chronic hepatitis patients, particularly chronic HCV patients and patients superinfected with more than one type of hepatitis virus. Results are summarized in Table 1.

TABLE 1

| Group | Sample Number | Positive |
|---|---|---|
| Super Normal | 100 | 0% |
| Normal | 96 | 0% |
| Disqualified | 172 | 2% |
| Hepatitis | 374 | 18% |
| Acute | 24 | 63% |
| Chronic HBV | 79 | 1% |
| Chronic HCV | 98 | 29% |
| Cryptogenic | 94 | 23% |
| Chronic HBV/HCV/HDV | 79 | 4% |

We claim:

1. A composition comprising isolated SVII virus.

2. The composition of claim 1, wherein said isolated SVII virus comprises a polynucleotide sequence shown in FIG. 1.

3. An isolated polynucleotide selected from the group consisting of, an isolated polynucleotide selectively hybridizable with a nucleotide sequence shown in FIG. 1,
a complement of an isolated polynucleotide selectively hybridizable with a nucleotide sequence shown in FIG. 1,
an isolated polynucleotide encoding a SVII protein or fragment of a SVII protein, and
a complement of an isolated polynucleotide encoding a SVII protein or a fragment of a SVII protein.

4. The isolated polynucleotide of claim 3, wherein said isolated polynucleotide is an antisense polynucleotide.

5. A composition comprising:
an isolated SVII protein or fragment thereof.

6. A vaccine composition comprising:
an isolated SVII protein or fragment thereof; and
a pharmaceutically acceptable excipient.

7. The vaccine composition of claim 6, further comprising an adjuvant.

8. An expression vector comprising an isolated polynucleotide encoding a SVII protein or a fragment of a SVII protein.

9. An expression vector comprising an isolated polynucleotide, wherein transcription of said isolated polynucleotide results in the production of an SVII antisense polynucleotide.

10. An isolated polyclonal antisera that specifically binds to a SVII virus or a protein thereof.

11. A monoclonal antibody which binds to a SVII virus or a protein thereof.

12. A method for detecting SVII virus, comprising:
contacting a sample with an antibody which specifically binds to SVII virus or a protein thereof; and
detecting complexes of said antibody and SVII virus or protein thereof.

13. A method for detecting SVII virus, comprising:
contacting a sample with a probe polynucleotide which selectively hybridizes to a SVII polynucleotide; and
detecting hybridization of said probe with a SVII polynucleotide.


SEQUENCE LISTING (5 sequences)

SEQ ID NO: 1 - DNA, 371 bp, Sentinel Virus II
gatcmggaaacgyttsgctcggtgcatgcagaaggacgggwtgaaggcggacgggattgacgacgacgacgacattgcgatgaaagatgggaccgcygacgtccttggcggggcggagcgcgagaaccaagacgacgaggacgaggacgtctacgcgcgcatccgtttccttcctgagcgggtatttgacacctccgcattgctgatcctgaagttctcgcttgcagacgctgattcagcgccgcttcgtcgcacctgctttggacgctgcaaaccgcacggctcggaccatcgtcagtttcctgcttcagaggtgaatttccgaccccgttggactttgctctctcttctctctctacccgacgacgatc

SEQ ID NO: 2 - DNA, 372 bp, Sentinel Virus II
  misc_feature (372)..(372): unknown can be a, t, g, c or there could be no nucleotide found in this position.
gatcgtcgtcgggtagagagagaagagagagcaaagtccaacggggtcggaaattcacctctgaagcaggaaactgacgatggtccgagccgtgcggtttgcagcgtccaaagcaggtgcgacgaagcggcgctgaatcagcgtctgcaagcgagaacttcaggatcagcaatgcggaggtgtcaaatacccgctcaggaaggaaacggatgcgcgcgtagacgtcctcgtcctcgtcgtcttggttctcgcgctccgccccgccaaggacgtcrgcggtcccatctttcatcgcaatgtcgtcgtcgtcgtcaatcccgtccgccttcascccgtccttctgcatgcaccgagcwaarcgtttcckgatcn

SEQ ID NO: 3 - protein, 123 aa, Sentinel Virus II
  Miscellaneous (14)..(14): This residue can be Met or Leu.
IRKRXARCMQKDGXKADGIDDDDDIAMKDGTADVLGGAERENQDDEDEDVYARIRFLPERVFDTSALLILKFSLADADSAPLRRTCFGRCKPHGSDHRQFPASEVNFRPRWTLLSLLSLPDDD

SEQ ID NO: 4 - protein, 124 aa, Sentinel Virus II
  Miscellaneous (111)..(111): This residue can be Ala or Pro.
DRRRVEREERAKSNGVGNSPLKQETDDGPSRAVCSVQSRCDEAALNQRLQARTSGSAMRRCQIPAQEGNGCARRRPRPRRLGSRAPPRQGRXRSHLSSQCRRRRQSRPPSXRPSACTEXXVSXS

SEQ ID NO: 5 - protein, 25 aa, Sentinel Virus II
  Miscellaneous (12)..(12): This residue can be Ser or Thr.
MSSSSSIPSAFXPSFCMHRAKRFXI